(19) United States
(12) Patent Application Publication
(10) Pub. No.: US 2003/0068340 A1
(43) Pub. Date: Apr. 10, 2003

(54) Parenteral formulations of 1-(5-tert-butyl-2-p-tolyl-2H-pyrazol-3-yl)-3-[4-(2-morpholin-4-yl-ethoxy)-naphthalen-1-yl]-urea

(75) Inventors: Michael L. Cappola, Wilton, CT (US); Susan Lynn Way, Danbury, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT (US)

(21) Appl. No.: 10/214,782

(22) Filed: Oct. 21, 2002

Related U.S. Application Data
(60) Provisional application No. 60/313,527, filed on Aug. 20, 2001.

(51) Int. Cl. A61K 9/00; A61K 31/724

(52) U.S. Cl. 424/400; 514/58

(57) ABSTRACT

Disclosed are formulations and compositions comprising, and process for preparing, improved parenteral dosage forms of 1-(5-tert-butyl-2-p-tolyl-2H-pyrazol-3-yl)-3-[4-(2-morpholin-4-yl-ethoxy)-naphthalen-1-yl]-urea. Also disclosed are methods of treating cytokine mediated diseases using such formulations and compositions.

### APPLICATION DATA

This application claims benefit to U.S. provisional application No. 60/313,527 filed Aug. 20, 2001.

### BACKGROUND OF THE INVENTION

1. Field of the Invention

The present invention relates to unique parenteral dosage formulations of 1-(5-tert-butyl-2-p-tolyl-2H-pyrazol-3-yl)-3-[4-(2-morpholin-4-yl-ethoxy)-naphthalen-yl]-urea, a pharmacological agent exhibiting novel anti-inflammatory activity. More particularly, the present invention relates to parenteral dosage formulations of 1-(5-tert-butyl-2-p-tolyl-2H-pyrazol-3-yl)-3-[4-(2-morpholin-4-yl-ethoxy)-naphthalen-1-yl]-urea that provide enhanced stability of the compound, improved solubility, and/or improved bioavailability, and are produced using unique process conditions.

2. Background of the Invention

1-(5-tert-butyl-2-p-tolyl-2H-pyrazol-3-yl)-3-[4-(2-morpholin-4-yl-ethoxy)-naphthalen-1-yl]-urea (hereinafter, “BIRB 796”) is disclosed in commonly assigned co-pending PCT Application No. PCT/US99/29165, herein incorporated by reference, as possessing unexpectedly significant inhibitory activity with respect to proinflammatory cytokines, such as tumor necrosis factor (TNF) and interleukin-1 (IL-1). BIRB 796 has

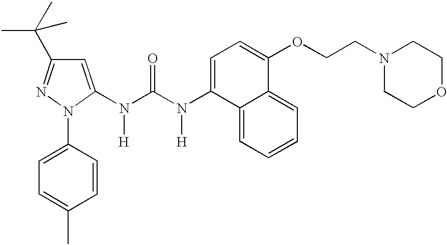

implications for the treatment of numerous cytokine mediates diseases including, but without limitation, arthritis including rheumatoid arthritis, psoriasis and Crohn's disease. BIRB 796 may be administered by the many routes of administration known in the art, including, but not limited to, orally, intravenously, intraperitoneally, intramuscularly, subcutaneously, bucally, rectally, aurally, ocularly, transdermally, etc.

While having many advantageous pharmacological properties, BIRB 796 has been found to possess certain less than desirable pharmaceutical properties, including poor solubility in many pharmaceutically-acceptable solvents and co-solvent solutions, and poor stability in solubilized form. Solubility and/or stability of BIRB 796 is very poor in most pharmaceutically-acceptable solvents that are used clinically. For example, after numerous tests it has been determined that BIRB 796 can not achieve a desirable solubility and/or stability in 30% PEG 400, 40% PG/10% ethanol or aqueous buffer solutions at any pH. Such solubility/stability problems translate into the commercial inability to make pharmaceutically-acceptable parenteral solutions of BIRB 796. As is understood by one of ordinary skill in the art, parenteral solutions of drugs are particularly advantageous when oral dosage forms can not be administered to a patient, such as when the patient is incapable of swallowing or taking the drug by mouth.

There is a need therefore for solubilized formulations of BIRB 796 with improved solubility and stability, which provide better bioavailability of the drug, as well as permit for the efficient preparation of the drug in solubilized form.

### SUMMARY OF THE INVENTION

The present invention discloses solubilized formulations of BIRB 796 for parenteral administration, and processes for manufacturing such formulations, that provide for improved stability and/or bioavailability of BIRB 796. In particular, advantageous liquid formulations of BIRB 796 are provided.

BIRB 796 has been determined to be poorly soluble in most pharmaceutically-acceptable solvents. Given the amount of BIRB 796 needed to be administered to provide for clinically-effective treatment, the volume of solvent(s) necessary to be administered parenterally may be clinically unacceptable. Typically, the greater the volume needed to be administered parenterally to a patient, the longer the infusion time, the higher the likelihood of a vehicle-related adverse effect, the more expensive the product is to produce, and the less likely that such drug will be found acceptable by the patient.

As disclosed herein, it has been discovered by the present inventors that the solubility of BIRB 796 in many solvents can be improved by incorporating oligosaccharides, and in particular substituted oligosaccharides, into the solvent mixture. Particularly advantageous oligosaccharides are the cyclodextrins, and in particular, the β-cyclodextrins, and yet more particularly the alkylated β-cyclodextrins (e.g., hydroxypropyl-β-cyclodextrin or HPBCD, and sulfobutylether-β-cyclodextrin or SBECD). The concentration of the cyclodextrin needed to effectuate solubilization depend on the type of solvent employed, the particular substituted cyclodextrin(s) utilized, and the conditions under which the solvent is maintained (temperature, pressure, etc.), as well as the concentration of the BIRB 796 in the solvent.

While improved solubilization of BIRB 796 can be achieved in pharmaceutically acceptable solvents by incorporating oligosaccharides, in particular substituted cyclodextrins, and more advantageously β-cyclodextrins, in such solvents (or solvent mixtures), it has further been found by the present inventors that solubilized forms of BIRB 796 often are unstable over extended periods of time in standard pharmaceutically-acceptable solvents. To avoid such a problem one could mix the drug substance with the oligosaccharide to form a dry powder which latter can be solubilized immediately prior to use. However, it has been found in practice that it generally requires several hours to solubilize a BIRB 796/β-cyclodextrin powder mixture to the extent that a clear solution is formed. Such method, of course, is disadvantageous in that it takes too long for efficient extemporaneous preparation.

The present inventors have found that the instability of BIRB 796 in solvents can be addressed in a unique fashion by freeze-drying the BIRB 796/oligosaccharide/solvent(s) mixture under controlled conditions to provide a stable dry powder for reconstitution. Such powder has been found to be easily reconstituted into a clear solution in several minutes, and to be stable at room temperature and at ambient pressures. This invention therefore also provides for effective pharmaceutical compositions containing BIRB 796 which can be used for treating cytokine mediated diseases.

### BRIEF DESCRIPTION OF THE DRAWINGS

The above description, as well as further objects, features and advantages of the present invention will be more fully understood with reference to the following detailed description when taken in conjunction with the accompanying drawings, wherein:

FIG. 1 is a graph of the solubility of BIRB 796 as a function of the concentration of hydroxypropyl-β-cyclodextrin (HPBCD);

FIG. 2 is a graph of the solubility of BIRB 796 as a function of the concentration of sulfobutylether-β-cyclodextrin (SBECD);

FIG. 3 is a graph of the binding isotherm for BIRB 796 and hydroxypropyl-β-cyclodextrin (HPBCD);

FIG. 4 is a graph of the binding isotherm for BIRB 796 and sulfobutylether-β-cyclodextrin (SBECD); and

FIG. 5 is a graph of the stability of BIRB 796 as a function of temperature and atmosphere composition.

### DETAILED DESCRIPTION OF THE INVENTION

The present invention overcomes many of the problems associated with the less than desirable solubility and stability of BIRB 796 in pharmaceutically-acceptable solvents. The present invention provides novel solubilized formulations of BIRB 796.

The present inventors have discovered that the solubility of BIRB 796 in many pharmaceutically-acceptable solvents can be improved by incorporation of oligosaccharides in the mixture, in particular substituted oligosaccharides. Significant enhancement is seen when cyclodextrins are used as the oligosaccharides, in particular when β-cyclodextrins are employed.

In a preferred embodiment of the present invention the BIRB 796 is solubilized in a pharmaceutically-acceptable solvent using one or more oligosaccharides. Preferably the oligosaccharide(s) utilized form an inclusion complex with the BIRB 796. The optimal amount of oligosaccharide in the solution depends on the particular oligosaccharide employed and its physical and physiological properties, the concentration of BIRB 796 to be placed in the solution, the ambient conditions (temperature, pressure, humidity), the particular solvent being employed, and the desired solution concentration. It is preferred that all solutions be prepared under sterile/aseptic conditions.

It is preferred that a biocompatible cyclodextrin, substituted or non-substituted, which provides suitable solubility and stability under the conditions encountered be used. Preferred cyclodextrins include the β-cyclodextrins, in particular sulfobutylether-β-cyclodextrin (SBECD) and hydroxypropyl-β-cyclodextrin (HPBCD). Additionally, any polymer, sugar, polyhydric alcohol, salt, salt combination, aqueous solvent, mixed aqueous and non-aqueous solvents, and the like, may be employed as a solubilizing adjunct if the compound is biocompatible and has sufficient product stability.

It is preferred that the oligosaccharide solution be prepared first, followed by dissolution of the BIRB 796 into the solution, although, less preferably, the BIRB 796 can be mixed into the solvent, and the oligosaccharide added into the BIRB 796-solvent thereafter. The resulting drug solution may then be stabilized for ambient shelf storage by drying the solution to a dry powder.

Drying may be performed in a single step, or in multiple steps, with the conditions of drying differing between steps. It is preferred that drying is performed under sterile/aseptic conditions. Drying of the solution is preferably under vacuum. A preferred method of drying is freeze-drying. Optimal freeze-drying conditions may change based on freeze-dryer design. As would be understood by one of ordinary skill in the art, other processes for drying the product in stable form may be employed other than freeze-drying. In addition to freeze drying, vacuum drying, spray drying and evaporative processes, without limitation, may be used for drying the product and making a stable product.

The resulting BIRB 796/oligosaccharide dried product, may be used clinically for any of the many uses being investigated for BIRB 796 including, but not limited to, rheumatoid arthritis, psoriasis, and Crohn's disease. The product may be sold as a human or veterinary prescriptive pharmaceutical. As set forth above, advantageously the oligosaccharides employed comprise cyclodextrin compounds, preferably β-cyclodextrin compounds. Unexpectedly good results in terms of dissolution have been obtained when cyclodextrin compounds, and in particular β-cyclodextrin compounds, are used in the solution medium.

It has been determined that the substituents on the cyclodextrin influence the solubility characteristics of the solution. For example, alkylated derivatives of β-cyclodextrin were found to solubilize BIRB 796 to a greater extent than none alkylated derivatives. As illustrated in Example I below, alkylated derivatives of β-cyclodextrin have been seen to provide significantly increased aqueous solubility as compared to underivatized β-cyclodextrin. While not limited by such hypothesis, it is believed such is due to differences in the complexation of the particular cyclodextrin with the BIRB 796.

### EXAMPLE 1

### Effect of β-cyclodextrin Substituents on BIRB 796 Solubility

Complexing cyclodextrin agents were evaluated as a means to improve the solubility of BIRB 796. The solubility of BIRB 796 was evaluated as a function of the concentrations of underivatized parent β-cyclodextrin (BCD), the alkylated derivatives hydroxpropyl-B-sulfobutylether-B-cyclodextrin (SBECD).

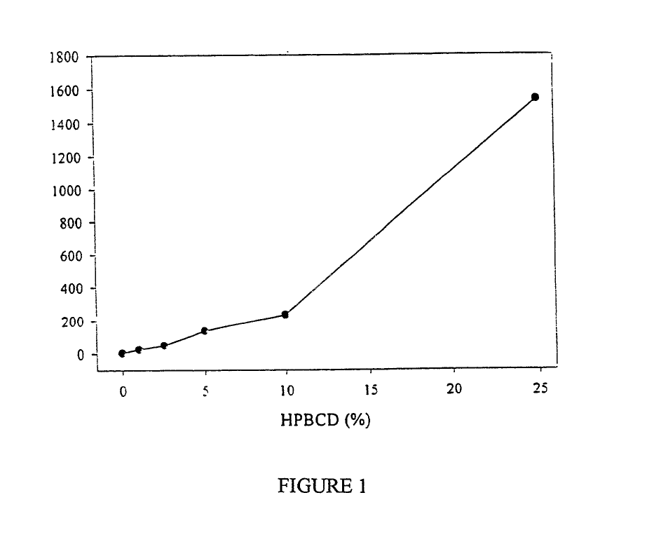

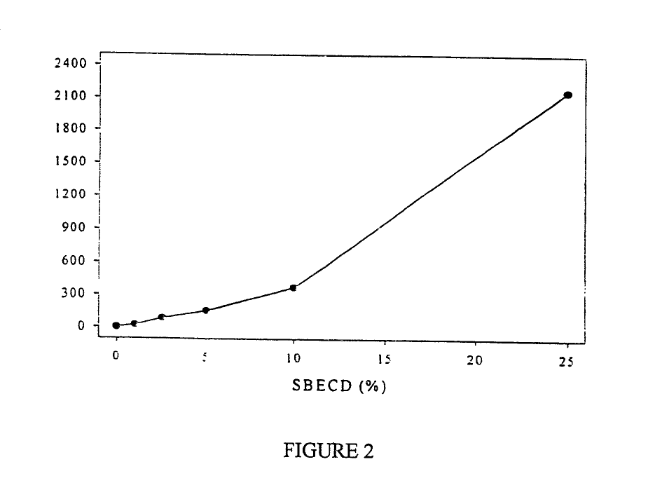

The solubility data for BIRB 796 in the presence of the cyclodextrins are presented in Table 1. The results of Table 1 with respect to the solubility of BIRB 796 as a function of HPBCD concentration are illustrated in FIG. 1, and the results of Table 1 with respect to the solubitlity of BIRB 796 as a function of SBECD concentration are shown in FIG. 2.

TABLE 1
Effect of Cyclodextrins on the Aqueous Solubility of BIRB 796

| | Conc. of | BIRB 796 |
|---|---|---|
| | Cyclodextrin | Solubility |
| Cyclodextrin | (% w/v) | (μg/mL) |
| — | 0 | 3.3 |
| BCD | 0.7 | 44 |
| | 1.4 | 74 |
| | 2 | 87 |
| HPBCD | 1 | 24 |
| | 2.5 | 49 |
| | 5 | 137 |
| | 10 | 233 |
| | 25 | 1535 |
| SBECD | 0 | 0.5 |
| | 1 | 21 |
| | 2.5 | 85 |
| | 5 | 156 |
| | 10 | 371 |
| | 25 | 2173 |

As discernable from Table 1, the alkylated derivatives (aqueous solubility<50% w/v) were seen to provide the advantage of greatly increased aqueous stability as compared to that for the underivatized BCD (aqueous solubility<2% w/v). The data as a whole show that the solubility of BIRB 796 increases as a function of cyclodextrin concentration. SBECD solubilized approximately 40% more than HPBCD at the highest concentration evaluated (25% w/v).

### Example II

### BIRB 796 Binding to Cyclodextrin

The HPBCD-BIRB 796 and SBECD-BIRB 796 interactions were shown to result in the formation of multiple species (non-linear, upward plot of solubility as a function of ligand concentration). The binding affinity for the BIRB 796 interaction with the ligands was evaluated in the following manner:

Sₜ=Sₒ+K₁₁Sₒ[L]+K₁₁K₁₂Sₒ[L]²

Lₜ=[L]+K₁₁Sₒ[L]+2K₁₁K₁₂Sₒ[L]²

where Lₜ (ligand) is the total HPBCD or SBECD concentration, Sₜ is the solubility of BIRB 796 in the presence of ligand, Sₒ is the solubility in the absence of ligand, K₁₁ is the binding constant for the 1:1 complex and K₁₂ is the binding constant for the 1:2 complex. Upon rearrangement, these two equations simplify to the following linear equation:

$$\frac{{S}_{t} - {S}_{o}}{{[ L ]}_{t}} = {K}_{11} {S}_{0} + {K}_{11} {K}_{12} {{S}_{0} [ L ]}_{t}$$

A plot of [L]ₜ vs. (SₜSₒ)/[Lₜ] yields the binding affinity according to the above equation.

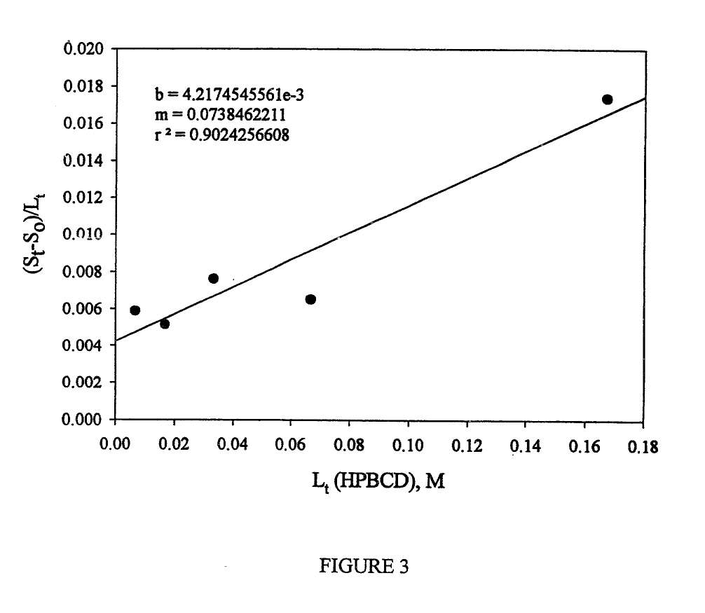

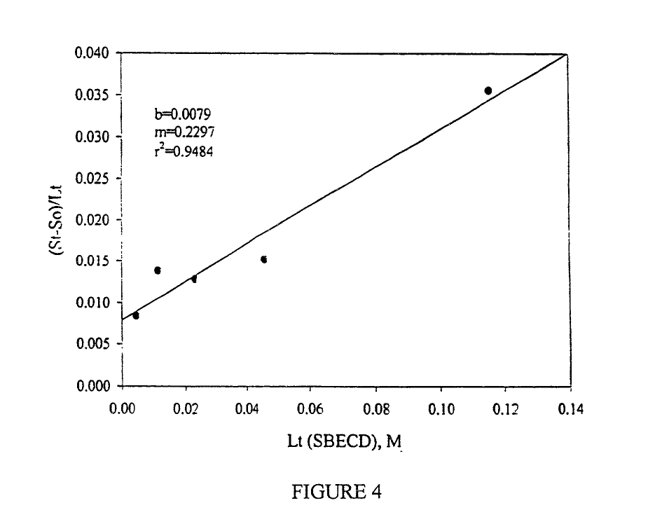

FIG. 3 illustrates the binding analysis for the interaction between BIRB 796 and HPBCD at different concentrations. FIG. 4 illustrates the binding analysis for the interaction between BIRB 796 and SBECD at different concentrations. The binding constants were obtained through the slope and intercept values. The binding affinities are presented in Table 2. The data in Table 2 show that SBECD had approximately an order of magnitude higher binding affinity for BIRB 796 than that for HPBCD.

TABLE 2
Binding parameters for BIRB 796 interaction with cyclodextrins

Binding Constants

| Cyclodextrin | K_{1:1} (M⁻¹) | K_{1.2} (M⁻¹) |
|---|---|---|
| HPBCD | 675 | 17.5 |
| SBECD | 8337 | 29.1 |

### Example III

### Stability of BIRB 796 in PEG 400 as Function of Temperature, Storage Atmosphere and Light Exposure

The stability of BIRB 796 in PEG 400 was evaluated. An initial BIRB 796 concentration of approximately 30 mg/mL was prepared and the percent of BIRB 796 remaining in the PEG 400 solvent evaluated over an approximately two week time frame for differing conditions. The PEG 400-BIRB 796 mixture was kept under several different conditions: (1) at room temperature (about 23° C.) in ambient air in the dark; (2) at room temperature (about 23° C.) in ambient air in the light; (3) at 40° C. in ambient air; (4) at 40° C. in ambient air, the mixture being imbued with 0.1% sodium metabisulfite, an antioxidant; (5) at 60° C. in ambient air; (6) at 60° C. in an oxygen atmosphere; and (7) at 60° C. in a nitrogen atmosphere.

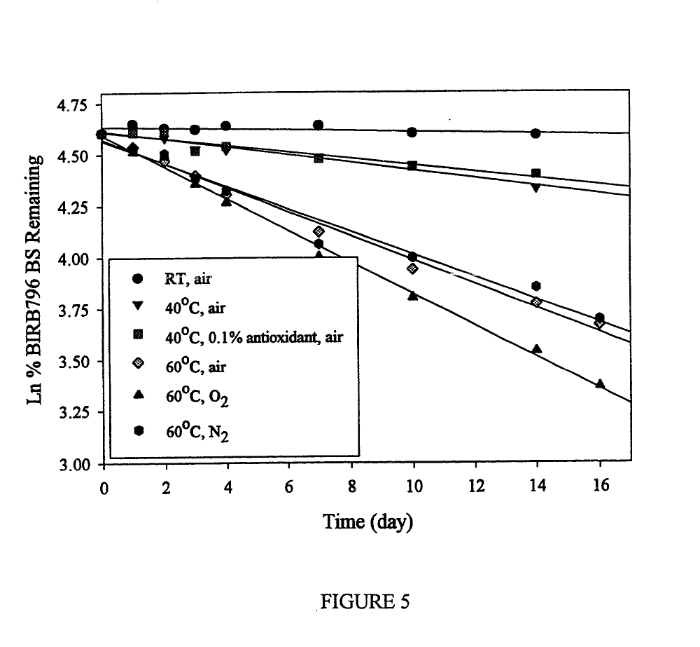

Turning to FIG. 5 there is shown graphically the effect of conditions 1 and 3-7 on the stability over BIRB 796 in PEG 400 over time. Degradation of BIRB 796 was seen to increase with increasing temperature. These data indicate that there is some difference in stability when conducted under different atmospheres (O₂, N₂ and air) as observed with the 60° C. data. Samples stored under oxygen atmospheres appeared to degrade faster than samples under nitrogen or air headspaces. Additionally, the presence of the antioxidant sodium metabisulfite (0.1%) appears to have had a slight effect over the time course observed with a trend toward higher recovery observed by the 14-day time point. However, there was almost no effect observed over the time prior to the terminal sample time. Perhaps if the study was carried out for a longer period of time, some additional benefit of using the antioxidant might have become apparent.

The BIRB 796 in PEG 400 mixture was stored both at room temperature (about 23° C.) in the dark (see FIG. 5), and under light. Samples stored under constant light conditions were found to change color over time. Analysis of the mixtures for BIRB 796 concentration over time found that samples stored under constant light degraded approximately three-times as fast as those stored under dark conditions. This result in conjunction with the result obtained with the sodium metabisulfite and nitrogen suggests that there are two paths of decomposition for BIRB 796 in PEG 400 solutions—oxidative and hydrolytic. Nitrogen and sodium metabisulfite appear to block the oxidative pathway (formation of N-oxide), but not the hydrolytic pathway. In addition, nitrogen was seen to block the formation of a dimer breakdown product found when the samples were stored at 60° C.

Table 3 below sets forth the calculated rate constants, half-life (days) and t₉₀ (days) from the data seen in FIG. 5, as well as with respect to the BIRB 796-PEG 400 sample which was stored at room temperature (23° C.) in ambient air under 24 hour light conditions. Rate constants were calculated using linear regression analysis of the data.

TABLE 3
Solution kinetics of degradation of BIRB 796 in PEG 400
(C₀ = 30 mg/mL)

| Condition | k_{obs} (day⁻¹) | t_{1/2} (days) | t₉₀ (days) |
|---|---|---|---|
| 23° C., air | −0.00253 | 274 | 41.5 |
| 23° C., air, light | −0.00780 | 88.8 | 13.5 |
| 40° C., air | −0.0192 | 36.1 | 5.47 |
| 40° C., 0.1% sodium metabisulfite | −0.0161 | 43.0 | 6.52 |
| 60° C., air | −0.0587 | 11.8 | 1.79 |
| 60° C., N₂ | −0.0552 | 12.6 | 1.90 |
| 60° C., O₂ | −0.0772 | 8.98 | 1.36 |

Half-life and T₉₀ data suggest that it would not be commercially practicable to manufacture BIRB 796 in PEG 400 for clinical use given the fact that significant decomposition was observed over two weeks even at 23° C. These solutions would not have the shelf-life needed for a commercially viable product.

### EXAMPLE IV

### Preparation of Freeze-Dried BIRB 796/Cyclodextrin

An aqueous solution of HPBCD or SBECD was prepared by dissolving the cyclodextrin in distilled water to a concentration of about 23% w/w. BIRB 796 was then added slowly into the solution with stirring. The resulting solution was stirred for a minimum of four (4) hours until the solution was clear. The clear solution was then filtered through a 0.22 μm membrane filter to remove any undissolved BIRB 796.

Eleven (11) milliliter samples of the resulting BIRB 796 solution were filled volumetrically into fifty (50) milliliter clear borosilicate serum vials. Butyl rubber stoppers were inserted into the vials for stoppering under vacuum. The vials were then placed on the shelf of a freeze dryer. The solution was frozen at −40° C. shelf temperature and the shelf temperature was held below −40° C. for approximately two (2) hours.

Primary drying was performed by cooling the condenser to less than −50° C. and then ramping the product to a shelf temperature of −10 to −15° C. The shelf temperature of −10 to −15° C. was maintained for about 30 hours. Secondary drying was accomplished by ramping the shelf temperature to 25° C., and holding the temperature at 25° C. for 4-16 hours. The final hold step was at 4° C. The product was stoppered under vacuum and removed.

The vials after removal from the chamber were crimped with aluminum seals and labeled.

### EXAMPLE V

### Stability of Freeze-Dried BIRB 796/Cyclodextrin

BIRB 796 (1 mg/ml) aqueous solutions containing 23% HPBCD or 23% SBECD were lyophilized in vials, and the vials were stored for various periods of time under one of several conditions: (1) 25° C./60% RH; (2) 40° C./75% RH or (3) 60° C./sealed-vial. Vials containing lyophilized BIRB 796 in either HPBCD or SBECD were removed and a solution of the same prepared at the appropriate time point and the reconstituted solution evaluated. Samples of the lyophilized powders were assayed using a stability-indicating HPLC assay. These data are presented in tables 4-5 below.

TABLE 4
Lyophilized BIRB 796 (1 mg/mL) in 23% SBECD

| | Storage | | |
|---|---|---|---|
| | time | % Assay | |
| Storage condition | (weeks) | (SD) | Number of Degradation peaks |
| Initial | 0 | 100.0 | NONE |
| 40° C./75% RH | 6 | 100.6 | NONE |
| | 12 | 102.4 | NONE |
| | 24 | 102.0 | NONE |
| 60° C. | 6 | 100.8 | NONE |
| | 12 | 102.8 | NONE |
| | 24 | 103.1 | NONE |
| | 12 | 103.8 | NONE |
| 25° C./60% RH | 24 | 104.3 | NONE |

TABLE 5
Lyophilized BIRB 796 (1 mg/mL) in 23% HPBCD

| | Storage | | |
|---|---|---|---|
| | time | % Assay | |
| Storage condition | (weeks) | (SD) | Number of Degradation peaks |
| Initial | 0 | 100 | NONE |
| 40° C./75% RH | 4 | 98.7 | 2 |
| | 8 | 100.0 | 2 |
| | 20 | 100.9 | 3 |
| 60° C. closed | 4 | 99.2 | 2 |
| | 8 | 100.0 | 2 |
| | 20 | 97.5 | 3 |
| 25° C./60% RH | 20 | 100.4 | 2 |

Based on the assay and the appearance of known degradation products, these data indicated that there was a clear improvement in chemical stability of the formulated product in the presence of both SBECD and HPBCD as compared to PEG 400. Additionally, the data indicate that there may be further benefit in using SBECD over HPBCD.

While the invention has been described with respect to preferred the art will readily appreciate that various changes and/or modifications can be made to the invention without departing from the spirit or scope of the invention as defined by the appended claims. All documents cited herein are incorporated in their entirety herein.

What is claimed is:

1. A pharmaceutical parenteral dosage formulation comprising:
(a) a pharmaceutically effective amount of 1-(5-tert-butyl-2-p-tolyl-2H-pyrazol-3-yl)-3-[4-(2-morpholin-4-yl-ethoxy)-naphthalen-1-yl]-urea;
(b) a pharmaceutically non-toxic amount of an oligosaccharide capable of forming an association or complex with 1-(5-tert-butyl-2-p-tolyl-2H-pyrazol-3-yl)-3-[4-(2-morpholin-4-yl-ethoxy)-naphthalen-1-yl]-urea in its aqueous state.

2. The pharmaceutical parenteral dosage formulation of claim 1 wherein said oligosaccharide contains amylose moieties.

3. The pharmaceutical parenteral dosage formulation of claim 1 wherein said oligosaccharides comprise a cyclodextrin.

4. The pharmaceutical parenteral dosage formulation of claim 1 wherein said oligosaccharides comprise a β-cyclodextrin.

5. A process of lyophilization, comprising:
a) providing the formulation of claim 1;
b) lyophilizing said formulation to create a lyophilized powder.

6. The process of claim 5, further comprising reconstitution of the lyophilized powder with a pharmaceutically-acceptable diluent to create a reconstituted solution.

7. The process of claim 6, wherein said the pharmaceutically-acceptable diluent comprises a diluent selected from the group consisting of: sterile water for injection, sterile saline for injection.

8. A lyophilized powder, produced by lyophilization of the formulation of claim 1.

9. A dry powder formulation for parenteral administration upon reconstitution with a diluent, said powder comprising 1-(5-tert-butyl-2-p-tolyl-2H-pyrazol-3-yl)-3-[4-(2-morpholin-4-yl-ethoxy)-naphthalen-1-yl]-urea and β-cyclodextrin.

10. A pharmaceutical composition comprising a pharmaceutically acceptable vehicle and a lyophilized 1-(5-tert-butyl-2-p-tolyl-2H-pyrazol-3-yl)-3-[4-(2-morpholin-4-yl-ethoxy)-naphthalen-1-yl]-urea and cyclodextrin fraction, said lyophilized fraction being produced by a process comprising:
(a) combining 1-(5-tert-butyl-2-p-tolyl-2H-pyrazol-3-yl)-3-[4-(2-morpholin-4-yl-ethoxy)-naphthalen-1-yl]-urea with a cyclodextrin compound in a pharmaceutically acceptable solvent;
(b) freezing the mixture of step (a) at a temperature of about −10° C. to about −196° C. to form a frozen fraction; and
(c) lyophilizing said frozen fraction to obtain a lyophilized 1-(5-tert-butyl-2-p-tolyl-2H-pyrazol-3-yl)-3-[4-(2-morpholin-4-yl-ethoxy)-naphthalen-1-yl]-urea and cyclo-dextrin fraction.

11. A pharmaceutical composition in the form of a dry powder comprising 1-(5-tert-butyl-2-p-tolyl-2H-pyrazol-3-yl)-3-[4-(2-morpholin-4-yl-ethoxy)-naphthalen-1-yl]-urea and cyclodextrin, wherein said dry powder contains less than 25% water.

12. A pharmaceutical composition of claim 11 said dry powder contains less than 10% water.

13. The pharmaceutical composition of claim 12 wherein the cyclodextrin is a substituted cyclodextrin.

14. The pharmaceutical composition of claim 12 wherein the cyclodextrin is a β-cyclodextrin.

15. A lypholized powder comprising 1-(5-tert-butyl-2-p-tolyl-2H-pyrazol-3-yl)-3-[4-(2-morpholin-4-yl-ethoxy)-naphthalen-1-yl]-urea and a cyclodextrin in a ratio of greater than 1:1.

16. The lypholized powder of claim 15 wherein the cyclodextrin is a substituted cyclodextrin.

17. The lypholized powder of claim 15 wherein the cyclodextrin is a β-cyclodextrin.

18. A dry powder comprising 1-(5-tert-butyl-2-p-tolyl-2H-pyrazol-3-yl)-3-[4-(2-morpholin-4-yl-ethoxy)-naphthalen-1-yl]-urea and a cyclodextrin in a ratio of greater than 1:1 made by the method comprising the steps of:
a. combining the cyclodextrin with a solvent to form a first solution;
b. admixing the 1-(5-tert-butyl-2-p-tolyl-2H-pyrazol-3-yl)-3-[4-(2-morpholin-4-yl-ethoxy)-naphthalen-1-yl]-urea into said first solution;
c. drying the resultant solution of step b.

19. A method for parenterally delivering 1-(5-tert-butyl-2-p-tolyl-2H-pyrazol-3-yl)-3-[4-(2-morpholin-4-yl-ethoxy)-naphthalen-1-yl]-urea comprising the steps of admixing a cyclodextrin and 1-(5-tert-butyl-2-p-tolyl-2H-pyrazol-3-yl)-3-[4-(2-morpholin-4-yl-ethoxy)-naphthalen-1-yl]-urea in a solvent, removing the solvent by drying to produce a dry powder formulation, and reconstituting the dry powder formulation with a pharmaceutically-acceptable solvent.

20. The method of claim 19 wherein the cyclodextrin is a substituted cyclodextrin.

21. The method of claim 19 wherein the cyclodextrin is a β-cyclodextrin.

22. A parenteral pharmaceutical formulation of 1-(5-tert-butyl-2-p-tolyl-2H-pyrazol-3-yl)-3-[4-(2-morpholin-4-yl-ethoxy)-naphthalen-1-yl]-urea which is stabilized against decomposition said formulation comprising 1-(5-tert-butyl-2-p-tolyl-2H-pyrazol-3-yl)-3-[4-(2-morpholin-4-yl-ethoxy)-naphthalen-1-yl]-urea in aggregation with stabilizing amounts of a cyclodextrin wherein said 1-(5-tert-butyl-2-p-tolyl-2H-pyrazol-3-yl)-3-[4-(2-morpholin-4-yl-ethoxy)-naphthalen-1-yl]-urea and cyclodextrin are present in a weight ratio of about 1:1 to about 1:1000.

23. The parenteral pharmaceutical formulation of claim 22 wherein the formulation is a lyophilized powder.

24. The parenteral pharmaceutical formulation of claim 22 wherein the cyclodextrin is a substituted cyclodextrin.

25. The parenteral pharmaceutical formulation of claim 22 wherein the cyclodextrin is a β-cyclodextrin.

26. A method of treating a cytokine mediated disease in a mammal comprising administering a therapeutically effective amount of a parenteral pharmaceutical formulation according to claims 1 or 22.

27. The method of claim 26 wherein the cytokine mediated disease is selected from rheumatoid arthritis, Crohn's disease and psoriasis.
